(19) United States
(12) Patent Application Publication
(10) Pub. No.: US 2002/0182612 A1
(43) Pub. Date: Dec. 5, 2002

(54) Therapeutics and diagnostics based on a novel IL-1beta mutation

(75) Inventors: Gordon W. Duff, Sheffield (GB); Francesco Saverio di Giovine, Ranmoor (GB)

Correspondence Address: FOLEY, HOAG & ELIOT, LLP, PATENT GROUP, ONE POST OFFICE SQUARE, BOSTON, MA, 02109, US

(73) Assignee: Interleukin Genetics, Inc.

(21) Appl. No.: 09/969,215

(22) Filed: Oct. 2, 2001

Related U.S. Application Data
(63) Continuation of application No. 09/247,874, filed on Feb. 10, 1999.

(51) Int. Cl. C12Q 1/68; C07H 21/04; C12P 19/34

(52) U.S. Cl. 435/6; 435/91.2; 536/24.3

(57) ABSTRACT

Methods and kits for detecting IL-1B allele 2 (+6912) and thereby determining a patient's susceptibility to developing an inflammatory disorders are described. Also provided are screening assays for identifying candidate therapeutics and therapeutic methods for treating inflammatory disorders.

### BACKGROUND OF THE INVENTION

Genetic testing (also referred to as “genetic screening” or “genotyping”) involves the analysis of an individual's genomic DNA (or a nucleic acid corresponding thereto) to identify a particular disease causing or contributing mutation or polymorphism, directly or based on detection of a mutation or polymorphism (a marker) that is in linkage disequilibrium with the disease causing or contributing gene.

Early indication of a genetic predisposition to a particular disease provides an opportunity for medical intervention before the development of clinically characteristic symptoms. In addition, sophisticated genetic testing can in many instances differentiate individual patients with subtle or clinically indistinguishable differences, facilitating a more customized therapy. In addition, identification of a mutation can provide a target for identifying drug candidates.

Diseases and conditions, both monogenic and polygenic, for which diagnostic or prognostic genetic tests exist include: Cystic Fibrosis, Gaucher's Disease, Huntington's Disease, Duchenne Muscular Dystrophy, hemophilias, thalassemias, Alzheimer's Disease, breast, ovarian and prostatic cancers and periodontal disease. This list continues to grow.

The IL-1 gene cluster is located on the long arm of chromosome 2 (2q13) and contains at least the genes for IL-1α (IL-1A), IL-1β (IL-1B), and the IL-1 receptor antagonist (IL-1RN) within a region of 430 Kb (Nicklin, et al., Genomics 19: 382-4 (1994)). The agonist molecules, IL-1α and IL-1β, have potent pro-inflammatory activity and are involved with the initiation of many inflammatory cascades. Their actions, often via the induction of other cytokines such as IL-6 and IL-8, lead to activation and recruitment of leukocytes into damaged tissue, local production of vasoactive agents, fever response in the brain and the hepatic acute phase response. The IL-1 receptor antagonist binds to the IL-1 receptor, but does not activate a signal. IL-1α and IL-1β proteins bind to type I and type II IL-1 receptors, but only the type I receptor transduces a signal to the interior of the cell. In contrast, the type II receptor may be surface bound or may be shed to become a soluble receptor. The bound type I receptor binds the agonist molecule but does not transduce a signal to activate the cell. The soluble receptors bind agonists and act as a decoy receptor. The receptor antagonist and the type II receptor, therefore, are both anti-inflammatory in their actions.

Inappropriate regulation of IL-1 genes and inappropriate levels of functional IL-1 proteins (IL-1 axis components) appears to play a central role in the pathology of many diseases and conditions. In addition, there appears to be stable inter-individual differences in the rates of production of IL-1-axis components, and some of this variation may be accounted for by genetic differences at IL-1-axis gene loci (Molvig, et al., Scand. J. Immunol. 27:705-16 (1988); Pociot, et al., Eur. J. Clin. Invest. 22: 396-402 (1992)). Thus, identification of IL-1-axis genes in an individual is useful for determining that individual's susceptibility to certain diseases and conditions and for DNA fingerprinting. This information is also useful in defining new targets for development of novel therapeutics.

Certain alleles of the IL-1 gene cluster have been shown to be associated with particular disease states, including coronary artery disease (International Patent Application No. PCT/US98/04725), osteoporosis (U.S. Pat. No. 5,698,399), periodontal disease (U.S. Pat. No. 5,686,246), nephropathy in diabetes mellitus (Blakemore, et al., Hum. Genet. 97(3): 369-74 (1996)), diabetic retinopathy (International Patent Application No. PCT/GB97/02790), alopecia areata (Cork, et al., J. Invest. Dermatol. 104(5 Supp.): 15S-16S (1995)), Graves disease (Blakemore, et al., J. Clin. Endocrinol. 80(1): 111-5 (1995)), systemic lupus erythematosus (Blakemore, et al., Arthritis Rheum. 37: 1380-85 (1994)), lichen sclerosis (Clay, et al., Hum. Genet. 94: 407-10 (1994)), ulcerative colitis (Mansfield, et al., Gastoenterol. 106(3): 637-42 (1994)), juvenile chronic arthritis, particularly chronic iridocyclitis (McDowell, et al., Arthritis Rheum. 38: 221-28 (1995)), psoriasis and insulin dependent diabetes in DR 3/4 patients (di Giovine, et al., Cytokine 7: 606 (1995); Pociot, et al., Eur J. Clin. Invest. 22: 396-402 (1992)).

Additionally, certain alleles from both of the IL-1 (33221461) haplotypes (33221461 and 44112332) have been shown to be in linkage disequilibrium with the disease causing alleles (See Cox et al., Am. J. Human Genet. 62:1180-1188 (1998) and International Patent Application No. PCT/GB98/01481).

### SUMMARY OF THE INVENTION

The invention is based on the identification of a functional polymorphism within the IL-1B gene. In one aspect, the invention features the new polymorphism, as well as assays for determining the presence of the polymorphism in a subject. In one embodiment, the method comprises the step of detecting in a nucleic acid sample obtained from a subject, the presence or absence of the IL-1B allele 2 (+6912) or a DNA sequence that is in linkage disequilibrium with IL-1B allele 2 (+6912). In preferred embodiments, the IL-1B allele 2 is detected by: 1) performing a hybridization reaction between the nucleic acid sample and a probe that is capable of hybridizing to an IL-1B allele; 2) sequencing at least a portion of the IL-1B allele; or 3) determining the electrophoretic mobility of the IL-1B allele or a fragment thereof (e.g. fragments generated by endonuclease digestion). In another preferred embodiment, the IL-1B allele is subject to an amplification step, prior to performance of the detection step. Preferred amplification steps are selected from the group consisting of: the polymerase chain reaction (PCR), the ligase chain reaction (LCR), strand displacement amplification (SDA), cloning, and variations of the above (e.g. RT-PCR and allele specific amplification). In a particularly preferred embodiment, the sample is hybridized with a set of primers, which hybridize 5′ and 3′ to +6912 of a sense or antisense sequence of IL-1B allele 2 or a DNA sequence that is in linkage disequilibrium with IL-1B allele 2 (+6912) and is subject to a PCR amplification.

In another aspect, the invention features kits for performing the above-described assays. The kit can include DNA sample collection means and a means for determining whether a subject carries IL-1B allele 2 (+6912). The kit may also comprise control samples or standards. Control samples used in the kits or methods of the invention may be positive or negative. Positive controls can include IL-1B allele 2 (+6912). Positive controls can also include alleles in linkage disequilibrium with these alleles. Negative controls can include IL-1B allele 1 (+6912) and other alleles of the IL-1B (+6912) marker or markers in linkage disequilibrium therewith. A kit may also include an algorithmic device for assessing identity matches. The algorithmic device may be used in conjunction with controls, or may be used independently of controls. The kits of the invention may also contain a variety of additional components such as DNA amplification reagent, a thermostable DNA polymerase, a DNA purification reagent, a restriction enzyme, a restriction enzyme buffer, a DNA sampling device, deoxynucleotides (dNTPs) and the like.

Information obtained using the assays and kits described herein (alone or in conjunction with information on another genetic defect or environmental factor, which contributes to the same disease) is useful for predicting whether a non-symptomatic subject is likely to develop a disease or condition, which is caused by or contributed to by IL-1B allele 2 (+6912) or for identifying among subjects who have some signs of disease, those more likely to be on an aggressive disease trajectory. In addition, the information alone or in conjunction with information on another genetic defect contributing to the same disease (the genetic profile of the disease) allows customization of therapy for the particular disease to the individual's genetic profile. For example, this information can enable a doctor to: 1) more effectively prescribe a drug that will address the molecular basis of the disease or condition; and 2) better determine the appropriate dosage of a particular drug.

The ability to target patient populations expected to show the highest clinical benefit, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

In another aspect, the invention provides assays for screening test compounds to identify IL-1β agonists and antagonists. In one embodiment, the screening assay comprises contacting a cell transfected with an IL-1B gene containing allele 2 (+6912) operably linked to an appropriate promoter with a test compound and determining the level of expression of the IL-1B gene in the cell in the presence and in the absence of the test compound, wherein increased production of IL-1β protein in the presence of the test compound indicates that the compound is an IL-1β agonist and decreased production of IL-1β protein in the presence of the test compound indicates that the compound is an IL-1β antagonist.

The invention also features transgenic non-human animals which include (and preferably express) a heterologous form of an IL-1B allele 2 (+6912). Such transgenic animals can serve as animal models for studying cellular and/or tissue disorders, which are caused or contributed to by IL-1β allele 2 (+6912) or for use in drug screening.

In yet a further aspect, the invention features methods for treating diseases or conditions which are caused or contributed to by the presence of an IL-1B allele 2 (+6912) in a subject. In one embodiment, the method comprises administering to the subject, a pharmaceutically effective amount of an IL-1β antagonist of the invention.

In another aspect, the invention features methods for treating a disease or condition, which could benefit from increased levels of IL-1β protein in a subject. In one embodiment, the method comprises administering to the subject a pharmaceutically effective amount of an IL-1β agonist of the invention.

Other features and advantages of the invention will be apparent from the following detailed description and claims.

### BRIEF DESCRIPTION OF THE DRAWINGS

FIG. 1 shows the nucleic acid sequence for IL-1B GEN X04500 (SEQ ID No.1)

FIG. 2 shows the nucleic acid sequence for the full-length IL-1B containing IL-1B allele 2 (+6912), i.e. a guanine at position 2 (SEQ ID No.2).

FIGS. 3A and 3B are graphs, which plot IL-1β production (ng/10⁶ monocytes) by peripheral blood mononuclear cells cultured with 100 ng/ml of LPS for 18 hrs (2A) and 9 hrs (2B) in relation to the IL-1B genotype of 58 volunteers.

FIG. 4 is a graph, which plots the accumulation of IL-1B mRNA in human monocytes at 8 hrs after activation with LPS (1 μg/ml).

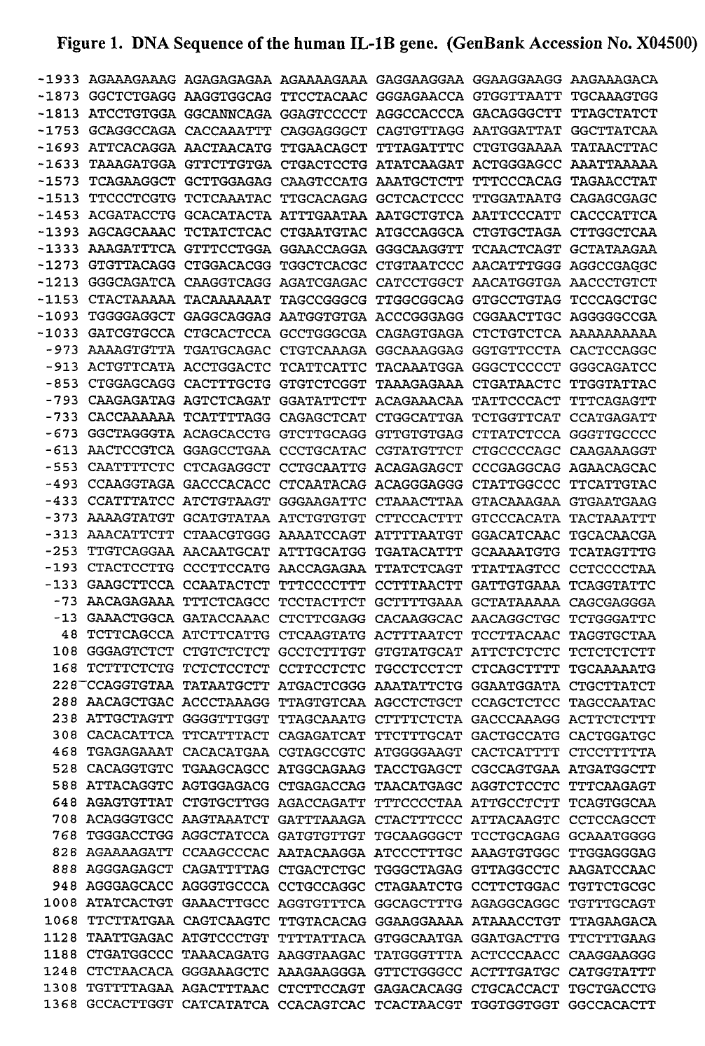

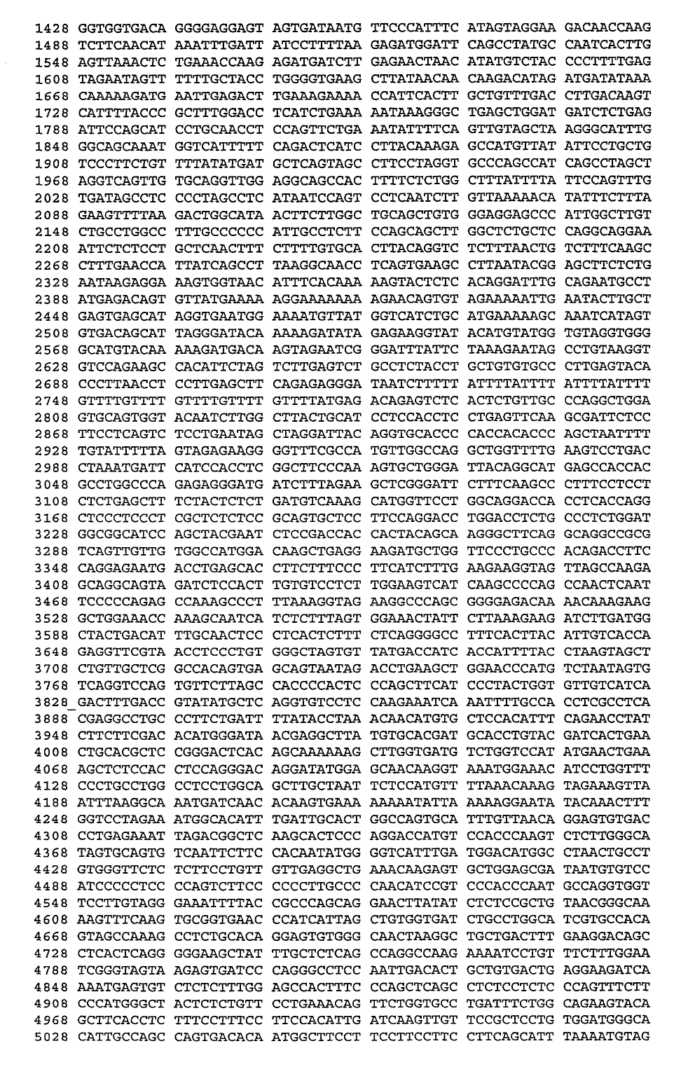

### DETAILED DESCRIPTION OF THE INVENTION

4.1 Definitions

For convenience, the meaning of certain terms and phrases employed in the specification, examples, and appended claims are provided below.

The term “allele” refers to the different sequence variants found at different polymorphic sites in DNA obtained from a subject. For example, IL-1B (+6912) has at least two different alleles. The sequence variants may be single or multiple base changes, including without limitation insertions, deletions, or substitutions, or may be a variable number of sequence repeats. Allelic variants at a certain locus are commonly numbered in decreasing order of frequency. In a biallelic situation the frequent allele is allele 1, the rarer allele will be allele 2.

2/2—Refers to the homozygous allele 2/allele 2 state.

2/1—Refers to the heterozygous allele 2/allele 1 state.

The term “allele detection means” refers to any means known to one of skill in the art for determining which allele is present at any given marker; for example, any means for determining whether the allele at position +6912 of the IL-1Bgene is allele 1, 2 or another allele. A variety of allele detection means are described herein.

The term “allelic pattern” refers to the identity of an allele or alleles at one or more polymorphic sites. For example, an allelic pattern may consist of a single allele at a polymorphic site, as for IL-1B (+6912) allele 1, which is an allelic pattern having at least one copy of IL-1B allele 1 at position +6912 of the IL-1B gene loci. Alternatively, an allelic pattern may consist of either a homozygous or heterozygous state at a single polymorphic site. For example, IL-1B (+6912) allele 2,2 is an allelic pattern in which there are two copies of the second allele at the +6912 marker of IL-1B and that corresponds to the homozygous IL-1B (+6912) allele 2 state. Alternatively, an allelic pattern may consist of the identity of alleles at more than one polymorphic site.

The term “antibody” as used herein is intended to refer to a binding agent including a whole antibody or a binding fragment thereof which is specifically reactive with an IL-1B polypeptide. Antibodies can be fragmented using conventional techniques and the fragments screened for utility in the same manner as described above for whole antibodies. For example, F(ab)₂ fragments can be generated by treating an antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an IL-1B polypeptide conferred by at least one CDR region of the antibody.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion of the IL-1 gene cluster. However, where the sample to be tested is genomic DNA, the control sample is preferably a highly purified sample of genomic DNA.

A “disease, which is caused by or contributed to by an IL-1 activity” refers to a disease that is caused by or contributed to by an over- or under-(insufficient) production of IL-1β in a subject. Examples of diseases which are caused or contributed to by an overproduction of IL-1β in a subject include inflammatory diseases, further including degenerative conditions, autoimmune diseases or trauma sequelae. Examples of diseases which are caused or contributed to by an under or insufficient production of IL-1β in a subject include cancers and infectious diseases.

The term “haplotype” refers to a set of alleles that are inherited together as a group (are in linkage disequilibrium). As used herein, haplotype is defined to include those haplotypes that occur at statistically significant levels (p_{corr}≦0.05). As used herein, the phrase “an IL-1 haplotype” refers to a haplotype in the IL-1 loci. At least two IL-1 proinflammatory haplotypes are known. The IL-1 (44112332) haplotype is associated with increased IL-1 α and β agonist activity, whereas the IL-1 (33441461) haplotype is associated with decreased IL-1 receptor antagonist activity.

The term “IL-1β agonist”, as used herein, is meant to refer to an agent that mimics or upregulates (e.g. potentiates or supplements) at least one bioactivity of an IL-1β protein. An agonist can be a wild-type gene, protein, peptide or derivative thereof having at least one bioactivity of the wild-type protein. An agonist can also be a compound (e.g. a small molecule) that upregulates expression of an IL-1B gene (e.g. increases the transcription rate or mRNA stability), or which increases at least one bioactivity of an IL-1β protein.

An “IL-1β antagonist” as used herein is meant to refer to an agent that downregulates (e.g. suppresses or inhibits) at least one bioactivity of an IL-1β protein. An antagonist can be a compound (e.g. an antisense or ribozyme molecule) that downregulates expression of an IL-1B gene or which reduces the amount of IL-1β protein present in a cell (e.g. an IL-1β antibody or other binding fragment that interferes with IL-1β binding to an IL-1β receptor).

The term “IL-1B allele (+6912)” refers to alternative forms of the IL-1B gene at marker +6912. “IL-1B allele 1 (+6912)” refers to a form of the IL-1B gene which contains a cytosine (C) at position +6912. “IL-1B allele 2 (+6912)” refers to a form of the IL-1B gene which contains a guanine (G) at position +6912. When a subject has two identical IL-1B alleles, the subject is said to be homozygous. When a subject has two different IL-1B alleles, the subject is said to be heterozygous. As shown herein, the IL-1B allele 2 (+6912) is a co-dominant “mutation”, i.e. one copy of the gene is sufficient to result in an altered phenotype relative to a wildtype allele.

The terms “IL-1 gene cluster” and “IL-1 loci” as used herein include all the nucleic acid at or near the 2q13 region of chromosome 2, including at least the IL-1A, IL-1B and IL-1RN genes and any other linked sequences. Nicklin et al., Genomics 19: 382-84, 1994. The terms “IL-1A”, “IL-1B”, and “IL-1RN” as used herein refer to the genes coding for IL-1, IL-1, and IL-1 receptor antagonist, respectively. The gene accession number for IL-1A, IL-1B, and IL-1RN are X03833, X04500, and X64532, respectively.

“Inflammatory disease” as used herein, refers to a disease or disorder that occurs in an individual due to tissue damage, regardless of the cause or etiology. This tissue damage can result from microbial invasion, autoimmune processes, tissue or organ allograft rejection, neoplasia, idiopathic diseases or such injurious external influences as heat, cold, radiant energy, electrical or chemical stimuli, or mechanical trauma. Whatever the cause, the ensuing inflammatory response is quite similar consisting of a complicated set of functional and cellular adjustments involving changes in microcirculation, movement of fluids, and influx and activation of inflammatory cells (e.g. leukocytes). Examples of such diseases and conditions include: coronary artery disease, osteoporosis, nephropathy in diabetes mellitus, alopecia areata, Graves disease, systemic lupus erythematosus, lichen sclerosis, ulcerative colitis, diabetic retinopathy, periodontal disease, juvenile chronic arthritis (e.g. chronic iridocyclitis), psoriasis, insulin dependent diabetes in DR 3/4 patients, asthma, chronic inflammatory liver disease, chronic inflammatory lung disease, lung fibrosis, liver fibrosis, rheumatoid arthritis, ulcerative colitis and other acute and chronic inflammation diseases of the Central Nervous System (CNS), gastrointestinal system, the skin and associated structures, the immune system, the hepato-biliary system, or any site in the body where pathology can occur with an inflammatory component.

Diseases or conditions, which could benefit from increased levels of IL-1B in a subject including infections (e.g. bacterial, fungal, viral or protistal infections), tumors and cancers.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage equilibrium”.

The term “marker” refers to a sequence in the genome that is known to vary among individuals. For example, the IL-1B gene has a marker at position +6912. The different sequence variants at a given marker are called alleles, mutations or polymorphisms. For example, the VNTR marker has at least five different alleles, three of which are rare. Different alleles could have a single base change, including substitution, insertion or deletion, or could have a change that affects multiple bases, including substitutions, insertions, deletions, repeats, inversions and combinations thereof.

The “non-human animals” of the invention include mammalians such as rodents, non-human primates, sheep, dog, horse, pig, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant genes is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues—as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts or products of combinatorial libraries, which can be screened with any of the assays of the invention to identify compounds that modulate an IL-1B bioactivity.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used to refer to DNA sequences, such as initiation signals, enhancers, and promoters, which induce or control transcription of protein coding sequences with which they are operably linked.

As used herein, the term “transgene” means a nucleic acid sequence which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

4.2 General

The invention is based, at least in part, on the identification of a novel allele at marker +6912 of the IL-1B gene (hereinafter referred to as the IL-1B allele 2 (+6912)). This cytosine (C) to guanine (G) transition occurs within the 3′ untranslated region (3′UTR) of the IL-1B gene (see SEQ ID No 2) and results in an increased level of IL-1β protein.

As described herein, individuals homozygous for the IL-1B allele 2 (+6912) accumulate approximately four times more immunoreactive IL-1β protein than homozygotes for allele 1 (+6912). In addition, the increased protein levels were found to be associated with increased steady state levels of mRNA. Thus, the increased level of IL-1β protein is presumably due to increased RNA stability and/or increased translation frequency.

Since IL-1β production is an early step in the inflammatory cascade, chronic, systemic overproduction of IL-1β in a subject predisposes that subject to development of certain diseases or disorders. The utility of detecting the IL-1B (+6912) marker as an indication of having a predisposition to the development of a disease, which is caused or contributed to by an overproduction of IL-1β in a subject is further supported by the finding described herein that this allele is tightly linked (99.36%) with the IL-1B (TaqI) allele 2 from marker +3954, which is part of the IL-1 (33221461) haplotype (See e.g. co-pending and coowned International Patent Application No. PCT/GB98/01481 and Cox et al., Am. J Human Genet. 62:1180-1188 (1998)).

In addition, the identification of the IL-1B allele 2 (+6912) and its involvement in IL-1β overproduction, enables screening assays for identifying IL-1β antagonists and agonists. IL-1β antagonists, which are identified as described herein, should prove useful for treating 30 diseases or conditions which are caused or contributed to by the presence of an IL-1B allele 2 (+6912) in a subject (e.g. inflammatory diseases). IL-1β agonists, on the other hand, should prove useful for treating diseases or conditions, which could benefit from increased levels of IL-1β protein in a subject (e.g. cancers and infections).

4.3 Predictive Medicine

4.3.1. Prognostic Assays and Kits

The present invention provides methods for determining whether a subject has or is likely to develop, a disease or condition that is caused by or contributed to by an abnormally high IL-1β level, by determining the transcription rate, stability or production level of an IL-1B nucleic acid or IL-1β protein.

In one embodiment, the method comprises determining whether a subject has an abnormally high transcription rate or an abnormally high mRNA and/or protein level, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the IL-1β protein or mRNA level is determined and compared to the level of IL-1β protein or mRNA level in a healthy subject.

In another embodiment, the method comprises measuring at least one activity of IL-1β. For example, the constant of affinity of an IL-1β protein of a subject with an IL-1β receptor can be determined. Comparison of the results obtained with results from similar analysis performed on IL-1β from healthy subjects is indicative of whether a subject has an normal endogenous level of IL-1β.

In preferred embodiments, the method is characterized as comprising detecting in a sample DNA obtained from a subject the existence of IL-1B allele 2 (+6912). In an exemplary embodiment, there is provided a nucleic acid composition comprising a nucleic acid probe including a region of nucleotide sequence which is capable of hybridizing to a sense or antisense sequence of the IL-1B allele 2 (+6912) For example, the nucleic acid is rendered accessible for hybridization, the probe is contacted with the nucleic acid of the sample, and the hybridization of the probe to the sample nucleic acid is detected. Such technique can be used to detect alterations or allelic variants at either the genomic or mRNA level as well as to determine mRNA transcript levels.

A preferred detection method is allele specific hybridization using probes overlapping IL-1B allele 2 (+6912) and having about 5, 10, 20, 25, or 30 nucleotides around the mutation or polymorphic region. In a preferred embodiment of the invention, several probes capable of hybridizing specifically to other allelic variants involved in the same disease are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to an IL-1β allele (+6912) under conditions such that hybridization and amplification of the IL-1β allele (+6912) occurs, and (iv) detecting the amplification product. These detection schemes are especially useful for the detection of nucleic acid molecules if such molecules are present in very low numbers.

In a preferred embodiment of the subject assay, IL-1β allele 2 (+6912) is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the IL-1β allele (+6912). Exemplary sequencing reactions include those based on techniques developed by Maxim and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labelled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on IL-1β allele 1 (+6912) is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify IL-1β allele 2 (+6912). For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control IL-1β alleles (+6912) are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of IL-1β alleles (+6912) in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting IL-1β alleles (+6912) and other alleles associated with the same disease (whether or not disease causing or contributing) include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect IL-1β alleles (+6912). For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized in the diagnostics described herein. In a preferred embodiment the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express the IL-1B gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Antibodies directed against IL-1β polypeptides may also be used to detect the level of IL-1β polypeptide expression. Protein from the tissue or cell type to be analyzed may easily be detected or isolated using techniques which are well known to one of skill in the art, including but not limited to western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of IL-1β polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-IL-1β polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Another embodiment of the invention is directed to kits for detecting a propensity for inflammatory disease in a patient. This kit may contain one or more oligonucleotides, including 5′ and 3′ oligonucleotides that hybridize 5′ and 3′ to the +6912 marker or detection oligonucleotides that hybridize to the +6912 marker directly. The kit may also contain one or more oligonucleotides capable of hybridizing near or at other alleles of the IL-1 gene cluster. PCR amplification oligonucleotides should hybridize between 25 and 2500 base pairs apart, preferably between about 100 and about 500 bases apart, in order to produce a PCR product of convenient size for subsequent analysis.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moities, metal binding moities, antigen or antibody moities, and the like. Oligonucleotides useful in the present invention are selected from the group consisting of any oligonucleotides that overlap or are contained in any of the following sequences:

| | |
|---|---|
| 5′GCTCCCACATTCTGATGAGCAAC3′ | (SEQ.ID.NO.3) |
| 5′TGCAGCACTCAGCAATGAGGAG3′ | (SEQ.ID.NO.4) |
| 5′CCCATTTAAATCTGAGCTTATATATTTTGAGT3′ | (SEQ.ID.NO.5) |
| 5′TCAATTTGGACTGGTGTGCTG3′ | (SEQ.ID.NO.6) |
| 5′TCAGAACCATTGAACAGTATGATATTTG3′ | (SEQ.ID.NO.7) |
| 5′ATCAAGTCCTTTAATTAACACTGAAAATATATAAGCTCAGAT3′ | (SEQ.ID.NO.8) |
| 5′AATCAAGTCCTTTAATTAAGAACTGAAAATATATAAGCTCAGATT3′ | (SEQ.ID.NO.9) |
| 5′AATCTGAGCTTATATATTTTCAGTCTTAATTAAAGGACTTGATT3′ | (SEQ.ID.NO.10) |
| 5′AATCTGAGCTTATATATTTTCAGTGTTAATTAAAGGACTTGATT3′ | (SEQ.ID.NO.11) |
| 5′CCGACTCGAGNNNNNNATGTGG3′ | (SEQ.ID.NO.12) |

The kit may, optionally, also include DNA sampling means such as the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood (such as SEQ. ID. NO. 12).

4.3.2. Pharmacogenomics

Knowledge of the particular IL-1B allele (+6912), alone or in conjunction with information on other genetic defects contributing to the same disease (the genetic profile of the particular disease) or on genetic factors that influence the kinetics or metabolism of a drug in question allows a customization of the therapy for a particular disease to the individual's genetic profile, the goal of “pharmacogenomics”. For example, subjects having IL-1B allele 2 (+6912) may be predisposed to developing an inflammatory disease. Thus, comparison of an individual's IL-1B profile to the population profile for the disease, permits the selection or design of drugs that are expected to be safe and efficacious for a particular patient or patient population (i.e., a group of patients having the same genetic alteration).

Pharmacogenomic studies can also be performed using transgenic animals. For example, one can produce transgenic mice, e.g., as described herein, which contain a specific IL-1B allele (+6912). The response of these mice to specific therapeutics can then be determined.

The ability to target populations expected to show the highest clinical benefit, based on the IL-1B or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of the IL-1B (+6912) marker is useful for optimizing effective dose).

The treatment of an individual with a particular therapeutic can be monitored by determining IL-1β protein level, mRNA level, and/or transcriptional level. This measurements will indicate whether the treatment is effective or whether it should be adjusted or optimized. Thus, IL-1B (+6912) can be used as a marker for the efficacy of a drug, e.g. during clinical trials.

In a preferred embodiment, the present invention provides a method for monitoring the effectiveness of treatment of a subject with an agent (e.g., an agonist or antagonist) of IL-1β (e.g. as identified by the screening assays described herein) comprising the steps of (i) obtaining a preadministration sample from a subject prior to administration of the agent; (ii) detecting the level of expression of an IL-1β protein, mRNA, or genomic DNA in the preadministration sample; (iii) obtaining one or more post-administration samples from the subject; (iv) detecting the level of expression or activity of the IL-1β protein, mRNA, or genomic DNA in the post-administration samples; (v) comparing the level of expression or activity of the IL-1β protein, mRNA, or genomic DNA in the preadministration sample with the IL-1β protein, mRNA, or genomic DNA in the post administration sample or samples; and (vi) altering the administration of the agent to the subject accordingly.

Cells of a subject may also be obtained before and after administration of a therapeutic to detect the level of expression of genes other than IL-1B, to verify that the therapeutic does not increase or decrease the expression of genes which could be deleterious. This can be done, e.g., by using the method of transcriptional profiling. Thus, mRNA from cells exposed in vivo to a therapeutic and mRNA from the same type of cells that were not exposed to the therapeutic could be reverse transcribed and hybridized to a chip containing DNA from numerous genes, to thereby compare the expression of genes in cells treated and not treated with the therapeutic.

4.4. Screening Assays for IL-1β Antagonists and Agonists

4.4.1 IL-1β Antagonists and Agonists

IL-1β antagonists and agonists can comprise any type of compound, including a protein, peptide, peptidomimetic, small molecule, or nucleic acid. An IL-1β antagonist or agonist can be a prescription drug or a nutrient.

For example, omega-3 polyunsaturated fatty acids and certain antioxidants are known to reduce IL-1β levels. Other antagonists can include antisense RNA, ssDNA or dsDNA, ribozymes and triplex molecules or repressor proteins that result in suppressed or decreased IL-1β production. Alternatively, the antagonist agent can interact with endogenous IL-1β protein or with a signal generating IL-1β receptor (e.g. a Type I receptor) preventing binding and initiation of signal transduction. For example, such an IL-1β antagonist can be an antibody or derivative thereof which interacts specifically with an IL-1β protein. Alternatively, the antagonist can be an IL-1β receptor ligand that binds to the receptor (thereby preventing endogenous IL-1β binding) but does not initiate signal transduction from the receptor. In an alternative embodiment, the antagonist is similar to those listed above, but targets the second, third and Nth nucleic acid or encoded protein in an IL-1β pathway.

“Antisense” therapy refers to administration or in situ generation of oligonucleotide molecules or their derivatives which specifically hybridize (e.g., bind) under cellular conditions, with the cellular mRNA and/or genomic DNA comprising IL-1B allele 2 (+6912), so as to decrease mRNA stability and/or the efficiency of translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes an IL-1β protein. Alternatively, the antisense construct is an oligonucleotide probe which is generated ex vivo and which, when introduced into the cell causes inhibition of expression by hybridizing with the mRNA and/or genomic sequences of an IL-1B gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the IL-1B nucleotide sequence of interest, are preferred.

Antisense approaches involve the design of oligonucleotides (either DNA or RNA) that are complementary to IL-1B mRNA. The antisense oligonucleotides will bind to the IL-1B mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. Antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their ability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotide can be prepared by use of controlled pore glass polymer supports (Sarin et al., 1988, Proc. Natl. Acad. Sci. U.S.A. 85:7448-7451), etc.

The antisense molecules can be delivered to cells which express IL-1β in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances.

Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous IL-1B transcripts and thereby prevent translation of the IL-1B mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive and can include but not be limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Ribozyme molecules designed to catalytically cleave IL-1B mRNA transcripts can also be used to prevent translation of IL-1B mRNA and expression of IL-1B (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy IL-1B mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

As in the antisense approach, the ribozymes can be composed of modified oligonucleotides (e.g., for improved stability, targeting, etc.) and should be delivered to cells which express the IL-1B gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous IL-1B messages and inhibit translation. Because ribozymes unlike antisense molecules, are catalytic, a lower intracellular concentration is required for efficiency.

Endogenous IL-1B gene expression can also be reduced by inactivating or “knocking out” the IL-1B gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional IL-1B (or a completely unrelated DNA sequence) flanked by DNA homologous to the endogenous IL-1β gene (either the coding regions or regulatory regions of the IL-1B gene) can be used, with or without a selectable marker and/or a negative selectable marker, to transfect cells that express IL-1B in vivo. Insertion of the DNA construct, via targeted homologous recombination, results in inactivation of the IL-1B gene.

Alternatively, endogenous IL-1B gene expression can be reduced by targeting deoxyribonucleotide sequences to form triple helical structures that prevent transcription of the IL-1B gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone mRNA.

4.4.2. Cell based or cell-free assays

The IL-1B mutation described herein facilitates the generation of cell-based assays, e.g., for identifying small molecule agonists or antagonists of IL-1β activity. For example, the screening assay comprises contacting a cell transfected with an IL-1B gene or fragment thereof, which contains IL-1B allele 2 (+6912) operably linked to an appropriate promoter with a test compound and determining the level of expression of the IL-1B gene in the cell in the presence and in the absence of the test compound, wherein increased production of IL-1β protein in the presence of the test compound indicates that the compound is an IL-1β agonist and decreased production of IL-1β protein in the presence of the test compound indicates that the compound is an IL-1β antagonist. A functional fragment suitable for use in the above described assays can be determined by one of skill in the art and may comprise, for example, 100, 250, 500, 750, 1000, 1250, 1500, 1750, 2000, 2250, 2500, 2750, 3000, 3250, 3500, 3750, 4000, 4250, 4500, 4750, 5000, 5250, 5500, 5750, 6000, 6100, 6200, 6300, 6400, 6500, 6600, 6700, 6800, 6900, or 7000 nucleotides.

4.4.4 Transgenic animals

Transgenic animals can also be made to identify IL-1β agonists and antagonists or to confirm the safety and efficacy of a candidate therapeutic. Transgenic animals of the invention include non-human animals containing a heterologous IL-1B allele 2 (+6912) under the control of an Il-1B promoter or under the control of a heterologous promoter. Such animals can be used, e.g., to determine the effect of higher endogenous levels of IL-1B protein. These animals can also be used to determine the effect of overexpression of IL-1B in a specific site in the animal, for identifying IL-1B agonists and antagonists or confirming their activity in vivo.

The transgenic animals can also be animals containing a transgene, such as reporter gene, under the control of an IL-1B promoter or fragment thereof. These animals are useful, e.g., for identifying drugs that modulate production of IL-1β, such as by modulating gene expression.

Methods for obtaining transgenic non-human animals are well known in the art. An IL-1B (+6912) transgene for generating the transgenic animals can encode the wild-type form of the IL-1β protein, or can encode homologs thereof, including both agonists and antagonists, as well as antisense constructs. In preferred embodiments, the expression of the IL-1B (+6912) is restricted to specific subsets of cells, tissues or developmental stages utilizing, for example, cis-acting sequences that control expression in the desired pattern. In the present invention, such mosaic expression of an IL-1β protein can be essential for many forms of lineage analysis and can additionally provide a means to assess the effects of, for example, expression level which might grossly alter development in small patches of tissue within an otherwise normal embryo. Toward this end, tissue-specific regulatory sequences and conditional regulatory sequences can be used to control expression of the IL-1B (+6912) in certain spatial patterns. Moreover, temporal patterns of expression can be provided by, for example, conditional recombination systems or prokaryotic transcriptional regulatory sequences. Genetic techniques, which allow for the expression of IL-1B (+6912) can be regulated via site-specific genetic manipulation in vivo, are known to those skilled in the art.

The transgenic animals of the present invention all include within a plurality of their cells a IL-1B (+6912) transgene of the present invention, which transgene alters the phenotype of the “host cell” with respect to regulation of cell growth, death and/or differentiation. In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation of expression of the IL-1B (+6912) transgene can be regulated via control of recombinase expression.

Use of the cre/loxP recombinase system to regulate expression of a IL-1B (+6912) transgene requires the construction of a transgenic animal containing transgenes encoding both the Cre recombinase and the subject protein. Animals containing both the Cre recombinase and a IL-1B (+6912) transgene can be provided through the construction of “double” transgenic animals. A convenient method for providing such animals is to mate two transgenic animals each containing a transgene.

One advantage derived from initially constructing transgenic animals containing a IL-1B (+6912) transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern.

Similar conditional transgenes can be provided using prokaryotic promoter sequences which require prokaryotic proteins to be simultaneous expressed in order to facilitate expression of the IL-1B (+6912) transgene. Exemplary promoters and the corresponding transactivating prokaryotic proteins are given in U.S. Pat. No. 4,833,080.

Moreover, expression of the conditional transgenes can be induced by gene therapy-like methods wherein a gene encoding the transactivating protein, e.g. a recombinase or a prokaryotic protein, is delivered to the tissue and caused to be expressed, such as in a cell-type specific manner. By this method, an IL-1B (+6912) transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2^{b}, H-2^{d} or H-2^{q} haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. Further, in such embodiments the sequence will be attached to a transcriptional control element, e.g., a promoter, which preferably allows the expression of the transgene product in a specific type of cell.

Retroviral infection can also be used to introduce the transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

4.5. Methods of Treatment

4.5.1. Diseases that can be Treated or Prevented By Administration of IL-1β Antagonists and Agonists

The invention also provides methods for preventing or treating in a subject, a disease or condition associated with abnormally high endogenous IL-1β levels (e.g. an inflammatory disease) based on administration of an effective amount of an antagonist; and methods for preventing or treating a disease or condition that could benefit from stimulation of the subject's immune system (e.g. an infection or cancer). Subjects at risk for such a disease can be identified by the prognostic assays described above.

4.5.2. Effective Dose

Toxicity and therapeutic efficacy of such compounds can be determined by standard pharmaceutical procedures in cell cultures or experimental animals, e.g., for determining The LD₅₀ (the dose lethal to 50% of the population) and the Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissues in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

4.5.3. Formulation and Use

Pharmaceutical compositions for use in accordance with the present invention may be formulated in a conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The present invention is further illustrated by the following examples which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLE 1

### Identification of the IL-1B 6912 Polymorphism

The region encompassing the AU-rich region of IL-1B (+6719/+7101 of GENBANK;X04500) was PCR amplified from 10 different individuals according to the following protocol:

A 403 bp fragment of the 3′UTR of the IL-1B gene was amplified from 100 ng of human genomic DNA. The primers and conditions were as follows:

| | |
|---|---|
| Forward primer: | 5′GCTCCCACATTCTGATGAGCAAC3′ (SEQ ID NO.3) |
| Reverse primer: | 5′TGCAGCACTCAGCAATGAGGAG3′ (SEQ ID NO.4) |
| Conditions: | 100 ng of template DNA using Taq polymerase (1U/100 μl |
| | reaction, Gibco) in 20 mM Tris-HCL (pH 8.4), 50 mM KCl, |
| | 2 mM MgCl₂, 0.2 mM each dNTP and 1 μM oligonucleotide |
| | primers. |
| Cycle: | 96° C., 5 min.; [95° C., 1 min.; 56° C., 1 min.; 72° C., 1 min.] × |
| | 35; 72° C., 5 min. |

To determine if novel polymorphisms existed in the 3 untranslated region (UTR) of the IL-1B gene, the PCR products generated above were sequenced as follows:

PCR products were purified by excision after electrophoresis through a 1% agarose gel, extraction by centrifugation through glasswool treated with 2-di chloromethylsilane at 6000 RPM for 10 minutes and ethanol precipitation. Five μg of the purified DNA was sequenced using dye-terminator cycle sequencing reagents and AMPLITAQ®POLYMERASE FS on an ABI 383 DNA sequencer according to manufacturer's instructions, and using the same primers that were used for the PCR amplification.

Extensive sequencing revealed a novel C to G transition polymorphism at position +6912, given in reference to the IL-1B coding nucleotides where +1 indicates the transcription start site of the sense strand. This allele is referred to hereinafter as IL-1B allele 2.

### EXAMPLE 2

### Population Screening of the +6912 Polymorphism

A cohort of blood donors from Northern England (n=820) were screened for this polymorphism using the methods described below, and the frequency of IL-1B allele 2 in this population was found to be 0.266. The allelic distribution of homozygotes and heterozygotes was in Hardy-Weinberg equilibrium (1/1=437; 1/2=330; 2/2=53).

Population screening for the frequency of each allele at the 6912 locus was carried out by the following two methods:

1. PCR-RFLP Method. This method was based on PCR amplification of the region surrounding the 6912 allele. Allele 2 can be identified by the lack of a HinfI restriction enzyme site. The PCR primers and conditions were as follows:

| | |
|---|---|
| Reverse Primer: | ′5CCCATTTAAATCTGAGCTTATATATTTTGAGT3′ (SEQ ID NO.5) |
| Forward Primer: | ′5TGCAGCACTCAGCAATGAGGAG3′ (SEQ ID NO.4) |
| Conditions: | 100 ng of template DNA using Taq polymerase (1U/100 μl |
| | reaction, Gibco) in 20 mM Tris-HCL (pH 8.4), 50 mM KCl, |
| | 2 mM MgCl₂, 0.2 mM each dNTP and 1 μM oligonucleotide |
| | primers. |
| Cycle: | 96° C., 5 min.; [95° C., 1 min.; 56° C., 1 min.; 72° C., 1 min.] × |
| | 35; 72° C., 5 min. |

The 203 bp PCR product was digested using HinfI (1U/34 μl reaction volume) at 37° C. for 8 hours. Digested samples were fractionated on 9% PAGE and visualized by ethidium bromide staining and UV transillumination. A band pattern of 89, 76, and 61 base pair fragments identifies the IL-1B allele 2, while 76, 61, 54, and 35 bp bands identify the IL-1B allele 1. Heterozygote DNA produced all five size fragments.

2. PCR 5′ Nuclease Method. Further screening was carried out using the TAQMAN®fluorogenic probe-based technique wherein a mismatched oligonucleotide probe spanning the allele is displaced, but a match oligonucleotide probe is digested by the 5′ nuclease activity of the TAQMAN® polymerase. These two states can be detected using probes that are differentially labeled with fluorogenic labels (e.g. tetra-chloro-carboxyfluoroscein (TET) or 6-carboxyfluoroscein (FAM)). The primers, probes and conditions were as follows:

| | |
|---|---|
| Forward Primer: | 5′TCAATTTGGACTGGTGTGCTC3′ (SEQ ID No.6) |
| Reverse Primer: | 5′TCAGAACCATTGAACAGTATGATATTTC3′ (SEQ ID NO.7) |
| Probe Allele 1: | 5′[TET]-ATCAAGTCCTTTAATTAACACTGAAAATATATAAGCTCAGAT3′ (SEQ ID NO.8) |
| Probe Allele 2: | 5′[FAM]-AATCAAGTCCTTTAATTAAGACTGAAAATATATAAGCTCAGATT3′ (SEQ ID NO.9) |
| Conditions: | PCRs were carried out in a final volume of 50 μl using 1U |
| | Taq polymerase of 7.5 mM MgCl₂, 0.2 mM dNTP's, 1 uM |
| | oligonucleotide primers, 10% glycerol, and a mixture of |
| | fluorogenic probes (30 to 40 nM). |
| Cycle: | [95′ C., 1 min.; 64° C., 1 min.] × 41. |

### EXAMPLE 3

### Cell Culture and IL-1β Protein Accumulation Studies

The possible association between this gene variant and IL-1 gene function was studied by isolating peripheral blood mononuclear cells from 58 volunteers and measuring their ability to produce IL-1β protein following cellular activation in vitro. The protocols were as follows:

Venous blood was obtained (100 ml) from 58 healthy male volunteers by venipuncture with a 19-gauge needle in 20 U/ml preservative-free calcium heparin (SANOFI WINTHROP LTD., Guilford, UK). Mononuclear cells were prepared by density gradient centrifugation (LYMPHOPREP™, Nycomed, Norway), washed free of contaminating platelets and total and differential counts of monocytes were performed. 5 mls of blood were used to obtain genomic DNA in order to genotype donors.

Mononuclear cells were cultured in RPMI 1640, 2% fetal calf serum, 50 mM L-glutamine, 100 U penicillin and 100 mg/ml streptomycin (all from GIBCO BRL, Paisley, Scotland). Cells (4-6×10⁶ monocytes/ml) were cultured in the presence or absence of 100 ng/ml lipopolysaccharide (LPS) from E. Coli, 0127:B8, SIGMA, Poole, UK).

Extracellular fractions were collected after 9 and 18 hrs. incubation (95% humidity, 5% CO₂) by centrifuging the plates and aspirating the clear supernatant. The cellular pellet was reconstituted to the original volume with fresh RPMI 1640, and lysed by addition of 9 mM CHAPS (SIGMA).

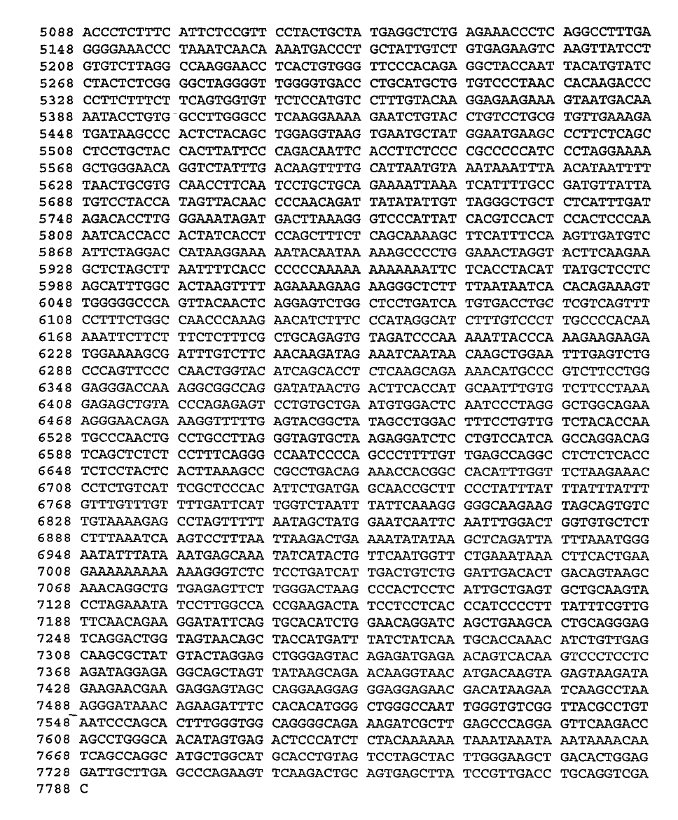

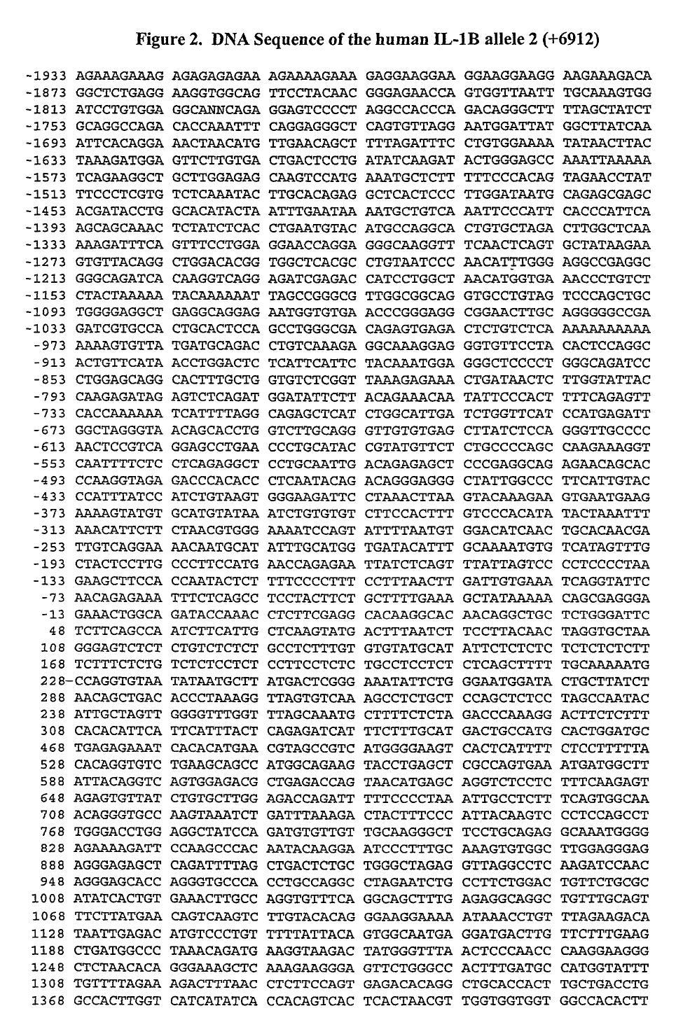

Both fractions (supernatant and cell-associated) were stored at −70° C. until tested for IL-1β content using a commercial ELISA (R&D EUROPE, Oxford, UK). The results were normalized for IL-1β content per 10⁶ monocytes. The data is presented in Table 1 and shown graphically in FIG. 2.

TABLE 1

| 1.1* | 1.2* | 2.2* | 6912 genotype | 1.1** | 1.2** | 2.2* |
|---|---|---|---|---|---|---|
| 4680 | 8020 | 8977 | | 8926 | 30000 | 18810 |
| 5290 | 6530 | 11685 | | 7799 | 15999 | 19560 |
| 1620 | 13110 | 8863 | | 8860 | 17361 | 21346 |
| 6128 | 6130 | 14103 | | 2644 | 17044 | 14103 |
| 5559 | 3133 | 17025 | | 12495 | 6498 | 26257 |
| 2727 | 4790 | | | 4108 | 8371 | |
| 2195 | 4376 | | | 1968 | 17169 | |
| 2998 | 8684 | | | 5588 | 31617 | |
| 100 | 11822 | | | 2845 | 19661 | |
| 1252 | 11420 | | | 4948 | 18404 | |
| 100 | 13640 | | | 6761 | 12445 | |
| 1417 | 7953 | | | 1781 | 3263 | |
| 1567 | 4040 | | | 3663 | 1721 | |
| 4668 | 5485 | | | 12016 | 2505 | |
| 5735 | 4732 | | | 9611 | 1896 | |
| 4529 | 7224 | | | 1738 | 2834 | |
| 8620 | 5790 | | | 11028 | 960 | |
| 8715 | 6458 | | | 4835 | 2747 | |
| 3615 | 7530 | | | 739 | 13628 | |
| 7021 | 6685 | | | 2723 | 17987 | |
| 4954 | 9633 | | | 1975 | 13871 | |
| 8307 | 15084 | | | 2830 | 17387 | |
| 5318 | 21343 | | | 1531 | 21343 | |
| 4500 | 6027 | | | 918 | 6027 | |
| 6838 | | | | 2498 | | |
| 14563 | | | | 11176 | | |
| 11446 | | | | 3869 | | |
| 4850 | | | | 4850 | | |
| 13198 | | | | 15156 | | |
| 5259 ± | 8318 ± | 12131 ± | mean + SEM | 5513 ± | 12531 ± | 12531 ± |
| 669 | 864 | 1560 | | 748 | 1807 | 1807 |

p = 0.0004 Kruskall-Wallis test p = 0.0002
data as pg/ml IL-1β total protein (intracellular + extracellular) accumulated by 10⁵ monocytes, stimulated with 100 ng/ml LPS,
*9 hrs time point.
**18 hrs time point.

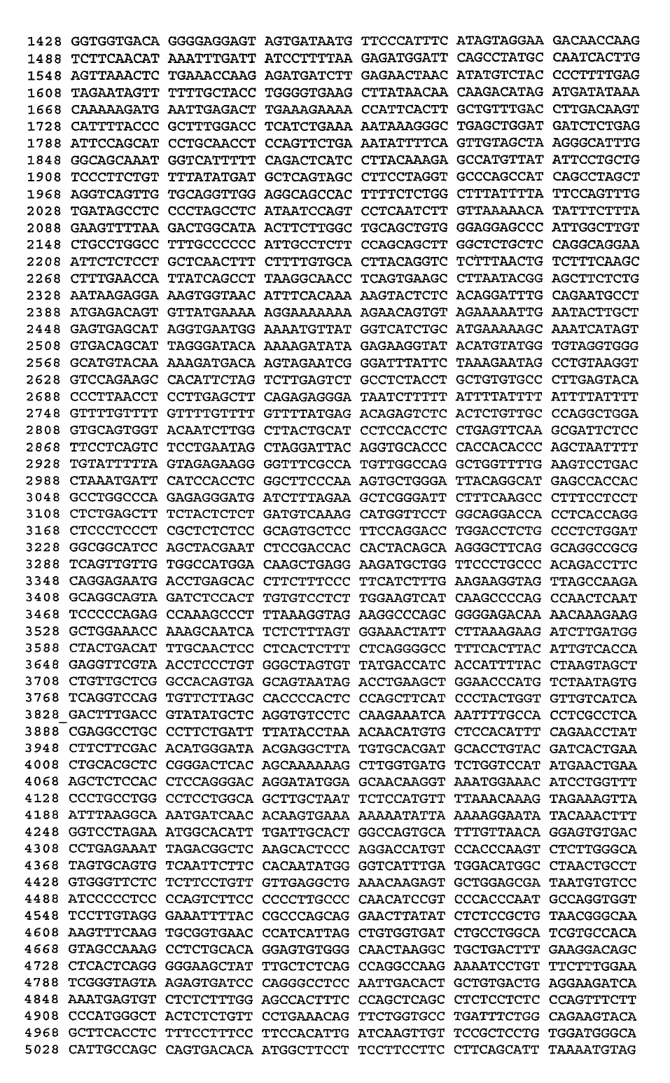

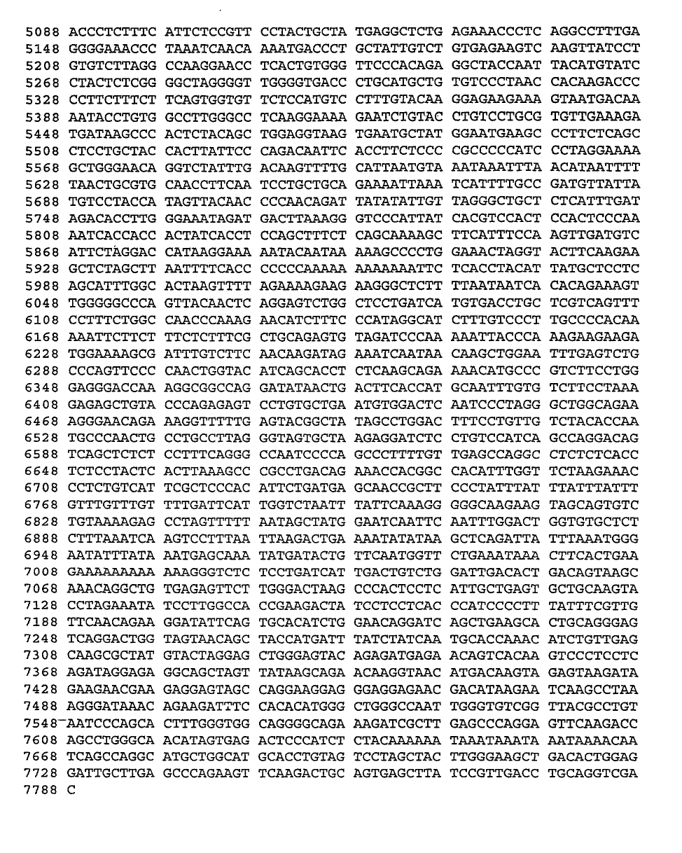

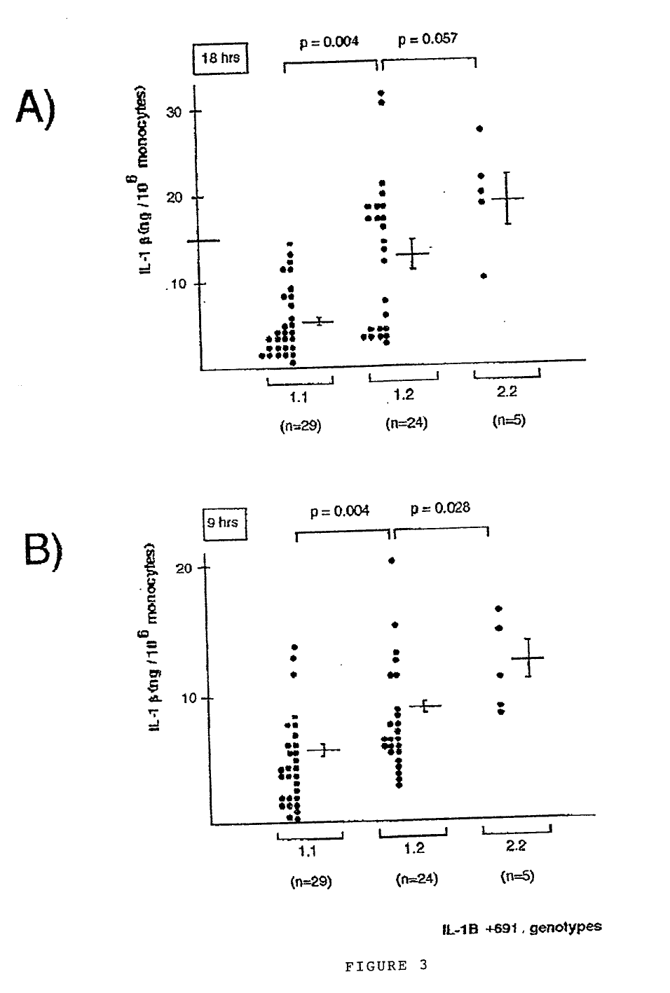

The data in Table 1 and FIG. 3 indicate that there is a large interindividual variation in response, and individuals homozygous for the IL-1B allele 2 (+6912) (2/2) accumulated approximately 4 times more immunoreactive IL-1β protein than homozygotes for allele 2 (+6912) (1/1).

### EXAMPLE 4

### Semi-Quantitative RT-PCR of IL-1B mRNA

To determine whether this increase in protein levels was associated with increased steady state levels of mRNA, a semi-quantitative transcription-PCR (RT-PCR) method was used to estimate levels of IL-1β mRNA in stimulated peripheral blood monocytes in vitro. The RT-PCR protocol was as follows:

Peripheral blood mononuclear cells were isolated by density centrifugation as described above from 9 healthy volunteers (four 2/2 homozygotes and five 1/1 homozygotes for +6912. Monocyte-enriched populations (>90% by morphology) were obtained by negative selection using anti-CD3 and anti-CD19 coated magnetic beads (DYNAL, Norway) according to the manufacturer's instructions. Cells (2×10⁷ monocytes per aliquot) were incubated in 1 ml RPMI 1640 (GIBCO, Paisley, Scotland) supplemented with 2 mM 1-glutamine, 100 U/ml penicillin, 100 μg/ml streptomycin and 10% low-endotoxin fetal calf serum. Following addition of LPS (1 μg/ml), cells were incubated at 37° C. (5% CO₂, 95% humidity) for 8 hours.

Monocytes were pelleted, washed twice in sterile saline, and resuspended by vigorous pipetting in 500 μl RNAzol® solution (CINNABIOTEX, TX). RNA was extracted according to the manufacturer's instructions.

Monocyte mRNA (1.5 μg/40 μl reaction) was reversed transcribed using poly(dT) primers and AMV reverse transcriptase (PROMEGA, US). Samples were incubated at 23° C. for 5 min., 42° C. for 2 hrs., and at 99° C. for 5 mins. Resulting mRNA/cDNA hybrids were stored at −20° C.

Reverse-transcribed transcripts were used as templates for two series of PCR reactions. Each series consisted of sequential 1:4 dilutions of the initial mRNA/cDNA hybrids and was amplified using a different set of PCR primers (one for IL-1β mRNA and the other for a control mRNA species—7B6—as described below). Each PCR reaction was reformed in duplicate using Taq polymerase (1U/100 μl reaction, GIBCO) in 20 mM Tris-HCL (pH 8.4), 50 mM KCl, 2 mM MgCl₂, 0.2 mM each dNTP and 1 μM oligonucleotide primers. The number of cycles needed to remain in suboptimal amplification range had been determined in preliminary experiments. The IL-1β PCR primers and conditions were as follows:

| | |
|---|---|
| Forward Primer: | 5′CTGCGTGTTGAAAGATGATAAGC3′ (SEQ ID NO. 13) |
| Reverse Primer: | 5′AAGTGAGTAGGAGAGGTGAGSGAGG3′ (SEQ ID NO. 14) |
| Conditions: | 100 ng of template DNA using Taq polymerase (1U/100 μl |
| | reaction, Gibco) in 20 mM Tris-HCL (pH 8.4), 50 mM |
| | KCl, 2 mM MgCl₂, 0.2 mM each dNTP and 1 μM |
| | oligonucleotide primers. |
| Cycle: | 96° C., 2 min.; [95° C., 1 min.; 56° C., 1 min.; 72° C., 30 |
| | sec.] × 22; 72° C., 5 min. |

The 7B6 PCR (7B6 is a stimulation-dependent, cell-cycle independent transcript) primers and conditions were as follows:

| | |
|---|---|
| Forward Primer: | 5′AGCCGTAGACGGAACTTCGC3′ (SEQ ID NO.15) |
| Reverse Primer: | 5′CTAAAACAGCGGAAGAGGT3′ (SEQ ID NO.16) |
| Conditions: | 100 ng of template DNA using Taq polymerase (1U/100 μl |
| | reaction, Gibco) in 20 mM Tris-HCL (pH 8.4), 50 mM |
| | KCl, 2 mM MgCl₂, 0.2 mM each dNTP and 1 μM |
| | oligonucleotide primers. |
| Cycle: | 96° C., 5 min.; [95° C., 1 min.; 56° C., 1 min.; 72° C., 1 min.] × |
| | 26; 72° C., 5 min. |

The final PCR products were added to 5 volumes of 0.6 M NaOH/10 mM EDTA, heated to 95° C. for 10 minutes, and each series was applied onto prewetted Zetaprobe™ membrane (positively charged nylon) by dot blotting. On each membrane were dotted a dilution series of IL-1β amplifications (or the corresponding series for 8B6) and two rows of linearised plasmid DNA containing the IL-1β and the 7B6 sequences being amplified. Following rinses with 0.4 M NaOH and 2×SSC, the membranes were baked at 80° C. for 1 hour to permanently fix the nucleic acid.

Membranes were pre-hybridized in SDS-Church buffer (7% SDS, 0.2 M disodium hydrogen orthophosphate, 0.1 M sodium dihydrogen orthophosphate). After 1 hr. incubation at 37° C., ³²P-end labeled oligonucleotide probes designed for IL-1β (5′CAGGACTCTCTGGGTACAGC3′—SEQ ID NO.17) or 7B6 (5′TCGTACTGTCTAGAGCTTGT3′—SEQ ID NO. 18) were added, and membranes left to hybridize for 16 hours at 37° C. Membranes were washed 3 times in SCC/Church buffer (330 mM NaCl, 90 mM sodium citrate, 16 mM di-sodium hydrogen orthophosphate, 8 mM sodium dihydrogen orthophosphate) at 37° C. before measuring hybridization by phosphoimaging.

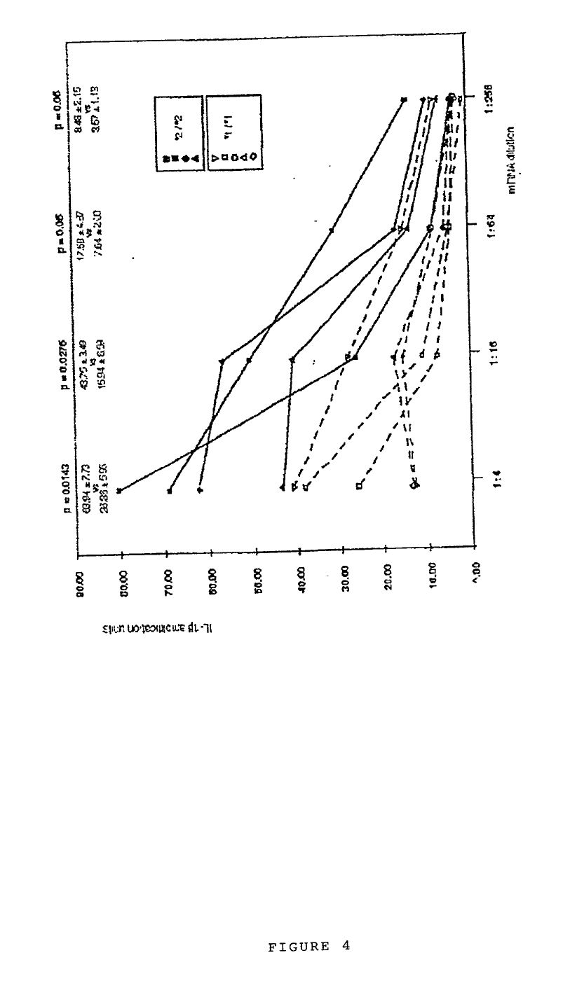

Raw data were normalized by the average counts on the dots containing the linearised plasmid, to avoid inter-membrane variation. Amplification units were defined by dividing IL-1β hybridization counts by 7B6 counts on the same mRNA preps, hence correcting for possible differences in cell numbers, mRNA/total RNA ratios, and loading errors generally. The data is shown in Table 2 and FIG. 4.

TABLE 2
IL-1B mRNA Accumulation
Table 2 A IL-B (+6912) 2/2 Monocytes

| | | | | 6911 | |
|---|---|---|---|---|---|
| *1:4 | 1:16 | 1:64 | 1:256 | genotype | donor |
| **64.832 | 49.280 | 30.101 | 13.601 | 2.2 | 2 |
| 38.055 | 36.256 | 12.194 | 10.418 | 2.2 | 5 |
| 72.227 | 82.022 | 23.279 | 13.579 | 2.2 | 7 |
| 70.286 | 23.956 | 7.548 | 3.282 | 2.2 | 9 |

| | | | | |
|---|---|---|---|---|
| 61.35 ± | 47.88 ± | 18.28 ± | 10.22 ± | mean ± |
| 7.9 | 12.5 | 5.14 | 4.9 | SEM |

Table 2 B IL-B(+6912) 1/1 Monocytes

| | | | | 6912 | |
|---|---|---|---|---|---|
| 1:4 | 1:16 | 1:64 | 1:256 | genotype | donor |
| 35.964 | 25.444 | 13.571 | 6.927 | 1.1 | 3 |
| 11.290 | 13.791 | 7.367 | 2.497 | 1.1 | 4 |
| 23.899 | 24.077 | 8.098 | 2.747 | 1.1 | 6 |
| 22.412 | 6.774 | 3.776 | 2.730 | 1.1 | 8 |
| 33.456 | 9.883 | 4.226 | 1.000 | 1.1 | 1 |

| | | | | |
|---|---|---|---|---|
| 25.40 ± | 15.99 ± | 7.41 ± | 3.18 ± | mean ± |
| 4.4 | 3.7 | 1.8 | 1.0 | SEM |

Table 2 C T-Student Test
Grouping Variable: IL-B (+6912) Genotype

| 1:4 | 1:16 | 1:64 | 1:256 | dilution |
|---|---|---|---|---|
| 0.004 | 0.030 | 0.063 | 0.022 | value |

*dilution of sample mRNA/cDNA hybrid used as template
**data expressed in amplification units, i.e. phosphorimager units from IL-1 B hybridization divided by 7B6, phosphorimager units in the same samples.

From Table 2 and FIG. 4, it is apparent that at 8 hrs. after cellular activation, cells from 2/2 individuals expressed significantly higher levels of IL-B mRNA than those from 1/1 homozygotes (p≦0.0002, approx. 2.5 times difference). These results showed that the observed differences in production of IL-1β protein that are associated with this base variation at (+6912) are also associated with differences in IL-1B mRNA accumulation.

### EXAMPLE 5

### Determination of Transcription Rate and mRNA Stability

Transcript accumulation is mainly influenced by transcription rate and mRNA stability, the first being determined classically by the 5′ (promoter) region of genes, the second, in most cytokine genes, by the 3′ UTR (AU-rich elements). The IL-1B (+6912) allelic variant is likely to determine differential IL-1B mRNA stability. This can be tested by two separate approaches. It is possible to measure the transcription rate of IL-1B in peripheral blood mononuclear cells of (1/1) and (2/2) homozygotes for (+6912) to determine if differences at this level can be excluded.

Analysis of transcription rates can be carried out by Nuclear run-off analysis. Nuclei are isolated from LPS stimulated PBMCs by Dounce homogenization followed by centrifugation through a sucrose gradient. Isolated nuclei are incubated with radio-labeled UTP for 30 minutes before being lysed and the RNA extracted. The labeled RNA is then used to probe a nitrocellulose membrane on which cloned cDNA for IL-1β, 7B6, and a negative control have been previously immobilized. The amount of radiolabeled probe bound to the membrane will reflect the rate at which the RNA was being transcribed at the time of the nuclear extraction.

The transcription rates should be roughly equivalent, and the mRNA accumulation is due to differential mRNA stability caused by the mutation in the 3′ UTR. Stable transfectants are established in monocytic cell lines, where the effects of the two allelic variants on reporter mRNA half-life can be readily established. IL-1B allele 2 (+6912) confers a longer half-life on the globin mRNA.

An alternative approach to studying differential mRNA stability can be based on in vitro degradation of synthetic human IL-1B mRNA. A human IL-1B cDNA is cloned into a bidirectional vector with an SP6 promoter capable of initiating RNA transcription. The construct contain the IL-1B coding sequence, followed by either IL-1B allele 1 (+6912) or IL-1B allele 2 (+6912). The cDNA is also added to cap and polyadenylation sites. The linearized vector is then used in a transcription reaction with radiolabeled nucleotides, and the resulting mRNA is purified by standard methods. Stability of this mRNA species is tested in vitro by incubating the two mRNA species with rabbit reticulocyte lysate mixed with monocytic cytoplasmic extracts, and extracting reaction products at different time points. Products are size fractionated by electrophoresis and quantified by phosporimaging. The rare allele (allele 2) will confer a longer half-life on the IL-1β mRNA.

### EXAMPLE 6

### Linkage With Other Alleles of the IL-1 (33221461) Haplotype

A number of genomes from North British Caucasians were genotyped for several biallelic markers of the IL-1 gene cluster by published methods, and concordancy with +6912 IL-1B genotypes calculated as percentage of individuals with matching genotypes. Individuals with partial matching scored 0.5; individuals with complete matching scored 1.0. Data are expressed in the following two-way tables:

TABLE 3

| | |
|---|---|
| Concordancy: | IL-1A +4845 |

| 85.58% | | 1/1 | 1/2 | 2/2 |
|---|---|---|---|---|
| IL-1B | 1/1 | 317 | 106 | 7 |
| +6912 | 1/2 | 54 | 239 | 35 |
| | 2/2 | 6 | 14 | 31 |

| | |
|---|---|
| Concordancy: | IL-1B −511 |

| 61.27% | | 1/1 | 1/2 | 2/2 |
|---|---|---|---|---|
| IL-1B | 1/1 | 150 | 199 | 70 |
| +6912 | 1/2 | 175 | 122 | 22 |
| | 2/2 | 27 | 18 | 2 |

| | |
|---|---|
| Concordancy: | IL-1B +3954 |

| 99.36% | | 1/1 | 1/2 | 2/2 |
|---|---|---|---|---|
| IL-1B | 1/1 | 125 | 1 | 0 |
| +6912 | 1/2 | 2 | 82 | 0 |
| | 2/2 | 0 | 0 | 21 |

| | |
|---|---|
| Concordancy: | IL-1RN +2018 |

| 64.64% | | 1/1 | 1/2 | 2/2 |
|---|---|---|---|---|
| IL-1B | 1/1 | 204 | 190 | 39 |
| +6912 | 1/2 | 206 | 109 | 12 |
| | 2/2 | 38 | 13 | 2 |

The data indicates that allele 2 is in tightly linked (99.36%) with the IL-1B(TaqI) allele 2 from the +3954 marker. The allele at +3953 is a member of the IL-1 (33221461) haplotype, and thus the IL-1B allele 2 (+6912) is in linkage disequilibrium with all of the alleles of the haplotype. From this it can be deduced that allele 2 is associated with, and probably contributes to the pathology of, at least diabetic retinopathy, periodontal disease, juvenile chronic arthritis, particularly chronic iridodyclitis, psoriasis and insulin dependent diabetes.

### EXAMPLE 7

### Disease Associated with the IL-1B Allele 2 (+6912)

Disease associations, as described in Example 6, can be confirmed by typing blood samples from diseased and non-diseased patients as described above.

What is claimed is:

1. A method for determining a subject's susceptibility to developing a disease or condition, which is caused by or contributed to by an inappropriately high level of IL-1β, comprising the steps of detecting an IL-1B allele (+6912) or an allele in linkage disequilibrium with an IL-1B allele (+6912) in a nucleic acid from the subject, wherein detection of IL-1B allele 2 (+6912) or an allele in linkage disequilibrium with IL-1B allele 2 (+6912) indicates that the patient has an increased susceptibility for developing a disease or condition, which is caused by or contributed to by an inappropriately high level of IL-1β.

2. A method of claim 1, wherein the disease or disorder is an inflammatory disease.

3. A method of claim 2, wherein said inflammatory disorder is selected from the group consisting of: coronary artery disease, osteoporosis, nephropathy in diabetes mellitus, alopecia areata, Graves disease, systemic lupus erythematosus, lichen sclerosis, ulcerative colitis, diabetic retinopathy, periodontal disease, juvenile chronic arthritis (e.g. chronic iridocyclitis), psoriasis, insulin dependent diabetes in DR 3/4 patients, asthma, chronic inflammatory liver disease, chronic inflammatory lung disease, lung fibrosis, liver fibrosis, rheumatoid arthritis and ulcerative colitis.

4. A method of claim 1, wherein the IL-1B allele (+6912) is detected by hybridizing the nucleic acid sample with at least one detection oligonucleotide that contains 6 consecutive nucleotides selected from the group consisting of: 5′ ATTAAC 3′; 5′ TTAACA 3′; 5′ TAACAC 3′; 5′ AACACT 3′; 5′ ACACTG 3′; 5′ CACTGA 3′; 5′ATTAAG 3′; 5′TTAAGA 3′; 5′ TAAGAC 3′; 5′ AAGACT 3′; 5′ AGACTG 3′; and 5′ GACTGA 3′.

5. A method of claim 1, wherein the IL-1B allele (+6912) is detected by contacting the sample DNA with a HinfI restriction enzyme and analyzing the restriction fragments, wherein a band pattern of 89, 76 and 61 base pair fragments identifies the IL-1β allele 2 and 76, 61, 54 and 35 bands identify the IL-1B allele 1.

6. A kit for determining a subject's susceptibility to developing a disease or condition, which is caused by or contributed to by an inappropriately high level of IL-1β, said kit comprising a first primer oligonucleotide that hybridizes 5′ or 3′ to an IL-1B +6912 allele or a marker that is in linkage disequilibrium with an IL-1B +6912 allele.

7. A kit of claim 6, which additionally comprises a second primer oligonucleotide that hybridizes 3′ to an IL-1B +6912 marker when the first primer hybridizes 5′ and hybridizes 5′ to an IL-1B +6912 marker when the first primer hybridizes 3′.

8. A kit of claim 6, wherein said first primer and said second primer hybridize to a region of an IL-1B gene that includes position +6912, wherein said region is in the range of between about 50 and 1000 base pairs.

9. A kit of claim 6 or 7, wherein said primers are selected from the group consisting of:
a) 5′GCTCCCACATTCTGATGAGCAAC3′(SEQ. ID. NO.2);
b) 5′TGCAGCACTCAGCAATGAGGAG3′(SEQ. ID. NO.3);
c) 5′CCCATTTAAATCTGAGCTTATATATTTTGAGT3′ (SEQ. ID. NO.4);
d) 5′TCAATTTGGACTGGTGTGCTC3′(SEQ. ID. NO. 5); and
e) 5′TCAGAACCATTGAACAGTATGATATTTG3′(SEQ. ID. NO. 6)

10. A kit of claim 9, further comprising a detection means, wherein said detection means is an appropriate amount of HinfI restriction enzyme to digest the sample and a means to analyse the digested sample, wherein a band pattern of 89, 76 and 61 base pairs identifies the IL-1B allele 2 and a band pattern of 76, 61, 54 and 35 identify the IL-1B allele 1.

11. A kit of claim 9, further comprising a detection means, wherein said detection means is a detection oligonucleotide that contains 6 consecutive nucleotides selected from the group consisting of: 5′ ATTAAC 3′; 5′TTAACA 3′; 5′TAACAC 3′; 5′AACACT 3′; 5′ACACTG 3′; 5′CACTGA 3′; 5′ ATTAAG 3′; 5′ TTAAGA 3′; 5′ TAAGAC 3′; 5′ AAGACT 3′; 5′ AGACTG 3′; and 5′GACTGA 3′.

12. A kit of claim 9, further comprising a DNA sampling means and a DNA sampling reagent.

13. A kit of claim 6, which further comprises a control.

14. A kit of claim 11, wherein said detection oligonucleotide includes a label.

15. A method for treating a subject for a disease or condition, which is caused by or contributed to by an inappropriately high level of IL-1β, comprising the steps of:
a) detecting an IL-1B allele (+6912) or an allele in linkage disequilibrium with an IL-1B allele (+6912) in a nucleic acid from the subject, wherein detection of IL-1B allele 2 (+6912) or an allele in linkage disequilibrium with IL-1B allele 2 (+6912) indicates that the patient has an increased susceptibility for developing a disease or condition, which is caused by or contributed to by an inappropriately high level of IL-1β; and
b) administering an IL-1β agonist to the subject.

16. A method of claim 15, wherein the disease or disorder is an inflammatory disease.

17. A method of claim 16, wherein said inflammatory disorder is selected from the group consisting of: coronary artery disease, osteoporosis, nephropathy in diabetes mellitus, alopecia areata, Graves disease, systemic lupus erythematosus, lichen sclerosis, ulcerative colitis, diabetic retinopathy, periodontal disease, juvenile chronic arthritis (e.g. chronic iridocyclitis), psoriasis, insulin dependent diabetes in DR 3/4 patients, asthma, chronic inflammatory liver disease, chronic inflammatory lung disease, lung fibrosis, liver fibrosis, rheumatoid arthritis and ulcerative colitis.

18. A method of claim 15, wherein the IL-1B allele (+6912) is detected by hybridizing the nucleic acid sample with at least one detection oligonucleotide that contains 6 consecutive nucleotides selected from the group consisting of: 5′ ATTAAC 3′; 5′ TTAACA 3′; 5′ TAACAC 3′; 5′ AACACT 3′; 5′ ACACTG 3′; 5′ CACTGA 3′; 5′ ATTAAG 3′; 5′ TTAAGA 3′; 5′ TAAGAC 3′; 5′ AAGACT 3′; 5′ AGACTG 3′; and 5′ GACTGA 3′.

19. A method of claim 15, wherein the IL-1B allele (+6912) is detected by contacting the sample DNA with a HinfI restriction enzyme and analyzing the restriction fragments, wherein a band pattern of 89, 76 and 61 base pair fragments identifies the IL-1B allele 2 and 76, 61, 54 and 35 bands identify the IL-1B allele 1.

20. A method for establishing an IL-1B (+6912) population profile in a specific population of individuals, comprising determining the IL-1B (+6912) genetic profile of the individuals in the population and establishing a relationship between IL-1B (+6912) genetic profiles and specific characteristics of the individuals.

21. A method of claim 20, wherein the specific characteristics of the individual include a susceptibility to developing a disease or condition, which is caused by or contributed to by IL-1β.

22. A method for identifying a compound that modulates IL-1B allele 2 (+6912) expression, comprising the steps of:
(a) contacting an appropriate amount of the compound with a cell or cellular extract, which expresses IL-1B allele 2 (+6912); and
(b) determining the resulting IL-1B expression level, wherein an increase or decrease in the IL-1B expression level in the presence of the compound as compared to the expression in the absence of the compound indicates that the compound is a modulator of IL-1B expression.

23. A method of claim 22, wherein the compound is an agonist.

24. A method of claim 22, wherein the compound is an antagonist.

25. A method of claim 22, wherein the compound is a member selected from the group consisting of a polypeptide, a nucleic acid, a peptidomimetic, and a small molecule.

26. A method of claim 25, wherein the nucleic acid is a member selected from the group consisting of an antisense, a ribozyme, and a triplex nucleic acid.

27. A method of claim 22, which additionally comprises the step of preparing a pharmaceutical composition from the compound.

28. A method of claim 22, wherein said cell is contained in an animal.

29. A method of claim 28, wherein the animal is transgenic.

30. A method of claim 29, wherein the IL-1B allele 2 (+6912) gene is human.

31. A compound identified by the method of claim 22.

32. A compound of claim 31, which is selected from the group consisting of: a small molecule, a polypeptide, a nucleic acid and a peptidomimetic.

33. A compound of claim 32, wherein the nucleic acid is selected from the group consisting of: an antisense molecule, a ribozyme and a triplex nucleic acid.

34. An isolated nucleic acid as shown in SEQ ID. No. 2.

35. An isolated nucleic acid of claim 34, which is comprised of between about 100 and about 7000 nucleotide and contains a guanine at position 6912.

36. An isolated nucleic acid of claim 34, which is comprised of between about 5000 and about 7000 nucleotides.

37. A transgenic non-human animal which contains and expresses an isolated nucleic acid of claim 34 in at least some of its cells.

38. A transgenic non-human animal of claim 37, which is heterozygous for the isolated nucleic acid of claim 34.

39. A transgenic non-human animal of claim 37, which is homozygous for the isolated nucleic acid of claim 34.

40. A transgenic non-human animal of claim 37, which exhibits a phenotype that is characteristic of an inflammatory disorder.

41. A transgenic non-human animal of claim 40, wherein said inflammatory disorder is selected from the group consisting of: coronary artery disease, osteoporosis, nephropathy in diabetes mellitus, alopecia areata, Graves disease, systemic lupus erythematosus, lichen sclerosis, ulcerative colitis, diabetic retinopathy, periodontal disease, juvenile chronic arthritis (e.g. chronic iridocyclitis), psoriasis, insulin dependent (Type I) diabetes, asthma, chronic inflammatory liver disease, chronic inflammatory lung disease, lung fibrosis, liver fibrosis, rheumatoid arthritis and ulcerative colitis and an arteritic disorder.

42. A method for identifying an agent as being an IL-1β antagonist, comprising administering the agent to a transgenic non-human animal of claim 37 and observing the effect on the animal's phenotype, wherein a amelioration of a phenotype characteristic of an inflammatory disorder indicates that the agent is an IL-1β antagonist.
